(19) United States
(12) Patent Application Publication
(10) Pub. No.: US 2020/0230082 A1
(43) Pub. Date: Jul. 23, 2020

(54) TOPICAL ACETAMINOPHEN FORMULATIONS FOR ITCH RELIEF

(71) Applicants: UNIVERSITY OF MIAMI, Miami, FL (US); Temple University-Of The Commonwealth System of Higher Education, Philadelphia, PA (US)

(72) Inventors: Gil YOSIPOVITCH, Miami, FL (US); Leigh NATTKEMPER, Miami, FL (US); David LEBO, Philadelphia, PA (US)

(21) Appl. No.: 16/630,139

(22) PCT Filed: Jul. 11, 2018
(86) PCT No.: PCT/US2018/041555

§ 371 Date: Jan. 10, 2020

Related U.S. Application Data
(60) Provisional application No. 62/531,221, filed on Jul. 11, 2017.

(51) Int. Cl. A61K 31/167; A61K 9/00; A61K 47/34; A61K 47/18; A61K 47/26; A61P 17/04

(52) U.S. Cl. CPC A61K 31/167; A61K 9/0014; A61P 17/04; A61K 47/18; A61K 47/26; A61K 47/34

(57) ABSTRACT

The present disclosure provides topical acetaminophen compositions. The present disclosure also provides method for treating acute and chronic itch using topical acetaminophen compositions.

### CROSS REFERENCE TO RELATED APPLICATIONS

The present application is a national phase of International Application No. PCT/US2018/041555, filed Jul. 11, 2018, which claims the benefit of priority to U.S. Provisional Application No. 62/531,221, filed Jul. 11, 2017, the contents of each of which are hereby incorporated by reference in their entirety for all purposes.

### FIELD OF THE DISCLOSURE

The present disclosure relates to topical acetaminophen formulations and uses thereof. More particularly, the disclosure relates to compositions and methods that are useful for treating acute and chronic itch.

### BACKGROUND

Pruritus, or itch, is a sensation that stimulates the desire or reflex to scratch, which can be either generalized or localized. It is a common distressing symptom, which may be caused by multifactorial etiologies. There is an unmet need to develop effective topical anti-itch medications for acute and chronic itch as currently there are only few topical formulations that have a direct effect on itch.

### SUMMARY OF THE DISCLOSURE

The present disclosure provides topical acetaminophen compositions and methods of treatment, which effectively diminish, alleviate, or otherwise prevent, symptoms such as itch.

Topical acetaminophen compositions disclosed herein comprise a therapeutically effective amount of acetaminophen and one or more pharmaceutically acceptable excipients. Exemplary excipients include solvents, gelling agents, thickening agents, occlusive agents, permeation enhancers, emulsifiers, pH adjusting agents, etc.

In some embodiments, topical compositions comprise from about 0.1% to about 5% acetaminophen.

In some embodiments, solvents used in the topical compositions comprise water, alcohol, polyethylene glycol (PEG), methoxypolyethylene glycol (MPEG), and/or mixtures thereof.

In some embodiments, thickening agents used in the topical compositions comprise hydroxypropylmethylcellulose (HPMC), carbomer, polyethylene glycol 6000, and/or mixtures thereof.

In some embodiments, occlusive agents used in the topical compositions comprise petrolatum, mineral oil, and/or mixtures thereof.

In one embodiment, topical compositions comprise a permeation enhancer. Exemplary permeation enhancers include oleic acid and propylene glycol.

In one embodiment, topical acetaminophen compositions comprise a therapeutically effective amount of acetaminophen, one or more solvents, and a thickening agent.

In other embodiments, topical acetaminophen compositions comprise a therapeutically effective amount of acetaminophen, one or more solvents, and an occlusive agent.

In various embodiments, a topical acetaminophen composition could be in the form of a gel, ointment, suspension, dispersion, cream, lotion, and paste.

In one embodiment, provided herein is a topical composition comprising, acetaminophen, a solvent selected from the group consisting of water, alcohol, polyethylene glycol (PEG), methoxypolyethylene glycol (MPEG), and mixtures thereof, and a thickening agent.

In another embodiment, provided is a topical composition comprising, acetaminophen, a solvent selected from the group consisting of propylene glycol, polyethylene glycol (PEG), methoxypolyethylene glycol (MPEG), and mixtures thereof, and an occlusive agent.

The present disclosure also provides methods for treating itch in a patient in need thereof, comprising administering to the patient an effective amount of a topical acetaminophen composition. The topical composition could be administered daily. The topical composition could be administered once, twice, 3 times, 4 times, or 5 times a day.

In some embodiments, the compositions and methods of the present disclosure reduce the intensity and duration of itch. In some other embodiments, the compositions and methods of the present disclosure provide peak itch relief about 5 to about 30 minutes after the administration of the composition.

Topical acetaminophen compositions disclosed herein could be used for treating acute or chronic itch.

### BRIEF DESCRIPTION OF THE DRAWINGS

FIG. 1 shows the picture of a gel. The gel is clear indicating that all the acetaminophen is completely dissolved.

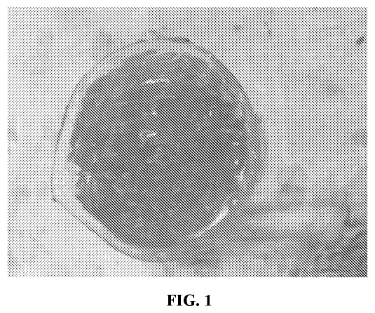

FIG. 2 shows the in vitro skin permeation of a gel composition containing 5% acetaminophen over 12 hours when tested using Franz cells not covered (non-occluded).

FIG. 3 shows the in vitro skin permeation of a gel composition containing 2.5% acetaminophen over 24 hours when tested using Franz cells not covered (non-occluded).

FIG. 4 shows the in vitro skin permeation of a gel composition containing 2.5% acetaminophen over 24 hours when tested using Franz cells covered (occluded).

FIG. 5 shows the in vitro skin permeation of a hydrophobic ointment composition containing 5% acetaminophen over 24 hours when tested using Franz cells covered (occluded).

FIG. 6 shows the in vitro skin permeation of a hydrophobic ointment composition containing 5% acetaminophen over 24 hours when tested using Franz cells not covered (non-occluded).

FIG. 7 shows the in vitro skin permeation of a hydrophilic base ointment composition containing 5% acetaminophen over 12 hours when tested using Franz cells not covered (non-occluded).

FIG. 8 shows a VAS itch intensity score in healthy subjects, administered with non-histaminergic itch stimuli, when treated with placebo vs topical acetaminophen composition.

### DETAILED DESCRIPTION

As referred to herein, all compositional percentages are by weight of the total composition, unless otherwise specified. As used herein, the word “include,” and its variants, are intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present technology, or embodiments thereof, may alternatively be described using more limiting terms such as “consisting of” or “consisting essentially of” the recited ingredients.

The present disclosure provides topical compositions comprising a therapeutically effective amount of acetaminophen and uses thereof.

The amount of acetaminophen present in the compositions can range from about 0.1% to about 5%, by weight, including values and subranges there between. For example, the amount of acetaminophen present in the compositions can range from about 0.1% to about 5%, about 0.3% to about 5%, about 0.5% to about 5%, about 0.8% to about 5%, about 1% to about 5%, about 1.5% to about 5%, about 2% to about 5%, about 2.5% to about 5%, about 1% to about 4%, about 1% to about 3.5%, about 1% to about 3%, about 2% to about 4%, about 0.1% to about 1%, about 0.1% to about 4%, about 0.1% to about 3%, about 0.5% to about 2.5%, by weight, including values and subranges there between.

In some embodiments, acetaminophen is present in the compositions in an amount of about 0.1%, 0.2%, 0.3%, 0.4%, 0.5%, 0.6%, 0.7%, 0.8%, 0.9%, 1%, 1.3% 1.5%, 1.8%, 2%, 2.3%, 2.5%, 2.8%, 3%, 3.3%, 3.5%, 3.8%, 4%, 4.3%, 4.5%, 4.8%, or 5%, by weight, including values and subranges there between.

In various embodiments, topical acetaminophen compositions comprise one or more solvents. The solvents include, but is not limited to, water, alcohol, polyethylene glycol (PEG), methoxypolyethylene glycol (MPEG), N-methylpyrrolidone, laureth-4, polyoxysorbitan-10, dethylene glycol monoethyl ether, DMSO, poloxamers, and/or mixtures thereof.

Suitable alcohols include C₁-C₆ alcohols including ethanol, isopropanol, and butanol, and diols such as propylene glycol (PG).

Polyethylene glycols that could be used as solvents in the compositions of the invention include, but are not limited to, PEG-200, PEG-300, PEG-400, PEG-500, PEG-600, upto PEG 3300. It is preferable that high molecular weight PEGs, e.g. PEG-600 to PEG-3300, be used in combination with low molecular weigh PEGs.

In some embodiments, poloxamers, in particular, liquid poloxamers, could be used as solvents. Poloxamers are well known in the pharmaceutical arts and are described, for example, by Irving R. Schmolka in “Poloxamers in the Pharmaceutical Industry,” in Polymers for Controlled Drug Delivery, Chapter 10 (Peter J. Tarcha ed., 1990). Poloxamers are triblock copolymers because they are composed of two different polymer blocks (i.e., hydrophilic poly(oxyethylene) blocks and hydrophobic poly(oxypropylene) blocks) configured as a triblock of poly(oxyethylene)-poly(oxypropylene)-poly(oxyethylene). Poloxamers are one class of block copolymer surfactants having a propylene oxide block hydrophobe and an ethylene oxide hydrophile. Poloxamers are commercially available (e.g., Pluronic® polyols are available from BASF Corporation). Alternatively, polaxamers can be synthesized by known techniques.

Aqueous formulations of poloxamers exhibit reverse thermal gelation, or reverse thermosetting. When an aqueous poloxamer formulation is heated over its gelation temperature, its viscosity increases and it transforms into a gel. When an aqueous poloxamer formulation is cooled below its gelation temperature, its viscosity decreases and it transforms into a liquid. The transition between gel and liquid does not involve a change in the chemical composition of the formulation, and is reversible and repeatable. The gel-liquid transition temperature of an aqueous poloxamer formulation can be adjusted by one of ordinary skill in the art using routine experimentation (e.g., by manipulating poloxamer concentration, pH and presence of other ingredients in the formulation). In some embodiments, compositions have a gelation temperature that is greater than the ambient temperature and less than or equal to the body temperature.

Methoxypolyethylene glycol (MPEG) that could be used as solvents in the compositions of the invention include, but are not limited to, MPEG-350 and MPEG-550.

Solvents may be present in an amount from about 5% to about 40%, about 10% to about 40%, about 5% to about 35%, about 5% to about 30%, about 10% to about 35%, about 15% to about 35%, about 20% to about 35%, or about 20% to about 40%, by weight, including values and subranges there between.

Propylene glycol may be present in an amount from about 5% to about 40%, about 10% to about 40%, about 5% to about 35%, about 5% to about 30%, about 10% to about 35%, about 15% to about 35%, about 20% to about 35%, or about 20% to about 40%, by weight, including values and subranges there between.

Ethanol or other lower chain alcohols may be present in an amount from about 5% to about 40%, about 10% to about 40%, about 5% to about 35%, about 5% to about 30%, about 10% to about 35%, about 15% to about 35%, or about 20% to about 35% by weight, including values and subranges there between.

In some embodiments, topical acetaminophen compositions comprise a therapeutically effective amount of acetaminophen, one or more solvents, and a thickening agent.

In another embodiment, topical acetaminophen composition comprises a therapeutically effective amount of acetaminophen, one or more solvents, and an occlusive agent.

Thickening agents include, but are not limited to, acacia, alginic acid, sodium alginate, bentonite, Carbopols® (now known as carbomers), carboxymethyl cellulose, ethylcellulose, gelatin, hydroxyethyl cellulose, hydroxypropyl cellulose, hydroxypropylmethyl cellulose (HPMC), methylcellulose, magnesium aluminum silicate (Veegum®), poloxamers (Pluronics®), polyvinyl alcohol, tragacanth, xanthan gum, stearic acid, sodium stearate and/or mixtures thereof.

In certain embodiments, thickening agents are present in the compositions in an amount from about 0.5% to about 10%, by weight, including values and subranges there between. For example, in various embodiments, thickening agents may be present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, by weight, including values and subranges there between. In some embodiments, thickening agents may be present in an amount from about 0.5% to about 5%, about 0.5% to about 4%, about 0.5% to about 3.5%, about 0.5% to about 3%, about 0.5% to about 2.5%, about 0.5% to about 2%, by weight, including values and subranges there between.

In some embodiments, topical acetaminophen compositions comprise a neutralizing or a pH adjusting agent. For example, in certain embodiments, the compositions comprise triethanolamine to raise the pH so that a carbomer can thicken a gel formulation.

In some embodiments, topical acetaminophen compositions comprise an occlusive agent. The occlusive agents include, but are not limited to, paraffin, petrolatum, mineral oil, ceresin wax, dimethicone, safflower oil, corn oil, coconut oil, and/or mixtures thereof.

The occlusive agents may be present in an amount from about 0.5% for agents like dimethecone to about 90% for agents like petrolatum.

In certain embodiments, topical acetaminophen compositions comprise a permeation enhancer. The permeation enhancers include, but are not limited to, oleic acid, propylene glycol, Laureth-4, N-methyl-pyrrolidone, Span 20, diethylene glycol mono ethyl ether, Lauryl-4, limonens, diethyl phthalate, triethyl citrate, triacetyl glycerin and bisabolol, and/or mixtures thereof.

The permeation enhancers may be present in an amount from about 0.5% to about 10%, by weight, including values and subranges there between. For example, in various embodiments, permeation enhancers may be present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%, by weight, including values and subranges there between. In some embodiments, permeation enhancers may be present in an amount from about 0.5% to about 5%, about 0.5% to about 4%, about 0.5% to about 3.5%, about 0.5% to about 3%, about 0.5% to about 2.5%, about 0.5% to about 2%, by weight, including values and subranges there between.

Topical acetaminophen compositions may comprise a viscosity reducing agent. The viscosity reducing agents include, but are not limited to, fatty acids including oleic acid, fatty alcohols, and oils, in particular, oils with high spreading.

The viscosity reducing agents may be present in an amount from about 0.1% to about 20%, by weight, including values and subranges there between. For example, in various embodiments, viscosity reducing agents may be present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 14%, 15%, 16%, 17%, 18%, 19%, or 20%, by weight, including values and subranges there between. In some embodiments, viscosity reducing agents may be present in an amount from about 0.5% to about 10%, from about 0.5% to about 15%, from about 5% to about 10%, from about 5% to about 15%, from about 1% to about 10%, from about 5% to about 20%, from about 0.5% to about 5%, by weight, including values and subranges there between.

In certain embodiments, topical acetaminophen compositions comprise an emulsifier. The emulsifiers include, but are not limited to, esters of sorbitan (known as Spans) including Span 80, Brij emulsifiers including Brij 30, Brij 60, etc., glycerol monosterate, stearic acid, sodium stearate, triethanolamine stearate, sodium lauryl sulfate, and/or mixtures thereof.

The emulsifiers may be present in an amount from about 2% to about 30%, by weight, including values and subranges there between. For example, in various embodiments, emulsifiers may be present in an amount of about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 14%, 15%, 16%, 17%, 18%, 19%, 20%, 21%, 22%, 23%, 24%, 25%, 26%, 27%, 28%, 29%, or 30%, by weight, including values and subranges there between. In some embodiments, emulsifiers may be present in an amount from about 1% to about 10%, about 1% to about 15%, about 1% to about 20%, about 5% to about 15%, about 5% to about 10%, about 5% to about 20%, about 5% to about 25%, about 5% to about 30%, about 10% to about 20%, about 10% to about 30%, about 15% to about 30%, including values and subranges there between.

The topical acetaminophen compositions could be prepared in a variety of forms. For example, the composition could be in the form of a gel, ointment, suspension, dispersion, cream, lotion, and paste.

In some embodiments, gel compositions may be used for immediate relief and ointments may be used for chronic use.

In some embodiments, acetaminophen is completely dissolved in a solvent before making the composition. The advantage of having acetaminophen completely dissolved in a solution is that it prevents variations in bioavailability that may arise from acetaminophen polymorphism.

In some embodiments, acetaminophen is completely dissolved in the composition and the composition is clear (e.g. no residues present) and/or transparent.

In some embodiments, topical acetaminophen compositions spread quickly on the skin. In some other embodiments, topical acetaminophen compositions dose acetaminophen to the epidermis quickly. In yet some other embodiments, topical acetaminophen compositions spread quickly and permeate through the skin fast leaving no residue when applied.

In some embodiments, topical compositions leave a film layer on the skin which does not allow water to leave the skin. The skin is more hydrated and therefore, more soft.

In some embodiments of topical acetaminophen compositions, polyethylene glycols may act like an ether when not in a moist environment and like an alcohol in the presence of a moist environment. That is, the composition is hydrophobic before applying to the skin and becomes hydrophilic with time once applied to the skin.

In some embodiments, topical acetaminophen compositions are completely or substantially free of preservatives. As used herein, the term “substantially free” means the composition does not comprise one or more preservatives at a concentration sufficient to have a stabilizing effect.

Solubility of acetaminophen (also referred to herein as APAP) in various solvents is known and is summarized in Table 1 below:

TABLE 1

| Solvent | Solubility % |
|---|---|
| Water | 1.7¹ |
| Ethanol | 37¹ |
| Propylene Glycol | 9.8² |
| PEG-400 | 18.9³ |

In some embodiments, a topical acetaminophen composition is prepared as a gel and comprises propylene glycol and ethanol as solvents to solubilize the acetaminophen.

An exemplary gel composition could be formulated as shown below:

TABLE 2

| | Conc % |
|---|---|
| Ingredient | (w/w) |
| APAP | 1 to 5 |
| Propylene Glycol | 10 to 30% |
| Denatured Ethanol | 10 to 30% |
| HPMC A | 0.5 to 2% |
| Carbomer | 0.3 to 1% |
| Triethanolamine | 0.1 to 2% |
| Purified water | qs |

In particular embodiments, a gel composition could be formulated as shown below:

TABLE 3

| | Concentration % |
|---|---|
| Ingredient | (w/w) |
| APAP | 1 to 5 |
| Propylene Glycol | 20 |
| Ethanol | 20 |
| HPMC (Methylcell) | 1 |
| Carbomer | 0.5 |
| Triethanolamine | 0.5 |
| Purified Water | QS |

In some embodiments, a gel composition may further comprise a permeation enhancer such as oleic acid.

In certain embodiments, a topical acetaminophen composition comprises no more than 20% propylene glycol and/or no more than 20% alcohol.

In some embodiments, a topical acetaminophen composition is prepared as an ointment and comprises propylene glycol and PEG-400 as solvents to solubilize the acetaminophen.

Exemplary ointment compositions could be formulated as shown in Tables 4 and 5 below:

TABLE 4

| | Conc % |
|---|---|
| Ingredient | (w/w) |
| APAP (Spectrum) | 1 to 5 |
| Propylene Glycol (Fisher Scientific) | 30 to 45% |
| Oleic Acid (Aldrich) | 1 to 10% |
| Petrolatum (Spectrum) | qs |

TABLE 5

| | Conc % |
|---|---|
| Ingredient | (w/w) |
| APAP (Spectrum) | 1 to 5 |
| PEG-400 (Spectrum) | 30 to 55% |
| Propylene Glycol (Fisher) | 10 to 30% |
| Light mineral oil (Spectrum) | 25 to 45% |
| SPAN 80 (Spectrum) | 0.5 to 2% |
| PEG-6000 (BASF) | 0.5 to 2% |

In one embodiment, an ointment formulation is formulated as shown in Table 6.

TABLE 6
Ointment Formulation # 1 Hydrophobic ointment

| | Concentration % |
|---|---|
| Ingredient | (w/w) |
| APAP | 0 to 5 |
| Propylene Glycol | 35 |
| Oleic Acid | 5 |
| Petrolatum | qs |

In another embodiment, an ointment formulation is formulated as shown in Table 7.

TABLE 7
Ointment Formulation # 2 Hydrophilic Base

| | Concentration % |
|---|---|
| Ingredient | (w/w) |
| APAP | 5 |
| PEG-400 | 40 |
| Propylene Glycol | 20 |
| Mineral Oil | 35 |
| Span 80 | 1 |
| PEG-6000 | 1 |

In some embodiments, a hydrophilic ointment composition may further comprise a permeation enhancer such as oleic acid.

The present disclosure also provides methods of administering topical acetaminophen compositions to a patient in need thereof. In one embodiment, a method for administering a topical acetaminophen composition to a patient in need thereof, comprises administering the composition topically on an area of a patient's skin.

In some embodiments, the present disclosure provides a method for treating pruritus or itch in a patient in need thereof, comprising administering a topical acetaminophen composition to the patient's skin.

The topical acetaminophen compositions can be administered daily. In some embodiments, the composition could be administered once, twice, 3 times, 4 times, or 5 times a day.

The topical acetaminophen compositions can be administered for treating acute episode of itch as well as chronic itch. In some embodiments, topical acetaminophen compositions can provide immediate relief.

In some embodiments, the compositions and treatment methods of the present disclosure may provide peak itch relief about 5 minutes, 10 minutes, 15 minutes, 20 minutes, 25 minutes, 30 minutes, 35 minutes, 40 minutes, 45 minutes, 50 minutes, 55 minutes, or 60 minutes, after the topical application of the composition.

In some other embodiments, the compositions and treatment methods of the present disclosure may provide peak itch relief about 5 to about 30 minutes, about 5 to about 25 minutes, about 5 to about 20 minutes, about 5 to about 15 minutes, or about 10 to about 30 minutes, after the topical application of the composition.

In some embodiments, the compositions and treatment methods of the present disclosure may reduce the duration of itch by about 2 minutes, 3 minutes, 4 minutes, 5 minutes, 6 minutes, 7 minutes, 8 minutes, 9 minutes, or by about 10 minutes.

In some other embodiments, the compositions and treatment methods of the present disclosure may reduce the duration of itch by about 2 to 10 minutes, about 2 to 7 minutes, about 1 to 5 minutes, about 1 to 10 minutes, about 2 to 5 minutes, about 2 to 4 minutes, about 2 to 3 minutes, about 3 to 5 minutes, about 3 to 6 minutes, about 4 to 8 minutes, about 4 to 10 minutes, or by about 5 to 10 minutes.

In some embodiments, the compositions and treatment methods of the present disclosure may reduce the intensity of itch by about 5%, 10%, 15%, 20%, 25%, 30%, 35%, 40%, 45%, 50%, 55%, 60%, 65%, 70%, 75%, or 80%.

In some other embodiments, the compositions and treatment methods of the present disclosure may reduce the intensity of itch by about 5 to about 50%, about 5 to about 45%, about 5 to about 40%, about 5 to about 30%, about 5 to about 25%, about 5 to about 20%, about 5 to about 15%, about 5 to about 10%, about 10 to about 50%, about 10 to about 40%, about 20 to about 50%, about 20 to about 40%, or by about 25 to about 50%.

The compositions and treatment methods of the present disclosure may also reduce the redness of the skin.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All patent and non-patent documents referenced throughout this disclosure are incorporated by reference herein in their entirety for all purposes.

### EXAMPLES

### Example 1: Acetaminophen Gel Composition

For each 100 ml:

| | |
|---|---|
| Acetaminophen (APAP) | 5 g or 2.5 g, |
| Propylene Glycol (PG) | 20% |
| Carbomer 980 | 0.5% |
| Triethanolamine | 0.5% |
| Methylcellulose | 1% |
| Denatured Alcohol | 20% |
| Purified Water | to 100 ml |

Materials: APAP, PG, triethanolamine, and methyl cellulose (MethylCell) were obtained from Spectrum Laboratory Products, Inc. Carbomer 980 (Carbopol® 980) was obtained from Dow Chemicals. Denatured alcohol was from Fisher Scientific and purified water was obtained from Baxter.

Preparation: 50 ml of water was heated to 75° C. Carbomer was added to the water with vigorous mixing. The APAP and PG were added to the alcohol. The mixture was heated to 75° C. and mixed. The water and alcohol mixtures were combined and triethanolamine was added during mixing. The liquid gel was placed in a sonic bath for 15 minutes. The gel formed during cooling.

### Example 2: Acetaminophen Hydrophobic Ointment

TABLE 8

| | Conc % |
|---|---|
| Ingredient | (w/w) |
| APAP | 5 |
| Propylene Glycol | 35 |
| Oleic Acid | 5 |
| Petrolatum | qs |

Materials: APAP and Petrolatum were obtained from Spectrum Laboratory Products, Inc. Propylene glycol was obtained from Fisher Scientific. Oleic acid was obtained from Sigma-Aldrich. Carbomer 980 (Carbopol® 980) was obtained from Dow Chemicals. Denatured alcohol was from Fisher Scientific and purified water was obtained from Baxter.

Preparation: APAP was dissolved in Propylene glycol. (Heat may be used if necessary.) Oleic Acid was added to this solution using levigation. This mixture was then incorporated into the petrolatum using geometric addition to the petrolatum.

### Example 3: Acetaminophen Hydrophilic Base Ointment

TABLE 9

| | Concentration % |
|---|---|
| Ingredient | (w/w) |
| APAP | 5 |
| PEG-400 | 40 |
| Propylene Glycol | 20 |
| Mineral Oil | 35 |
| Span 80 | 1 |
| PEG-6000 | 1 |

Materials: APAP, PEG-400, Light Mineral Oil, and Span 80 were obtained from Spectrum. Propylene glycol was obtained from Fisher Scientific. PEG-6000 was obtained from BASF.

Preparation: APAP was dissolved into propylene glycol with heat. Mineral Oil and Span were levigated together. The Mineral oil and span were mixed with the APAP and propylene glycol. PEG-400 and PEG-6000 were combined and made to dissolve with mild heat. This mixture was levigated with the APAP, Mineral Oil, Propylene Glycol and Span Mixture.

### Example 4: In Vitro Skin Permeation Studies of Acetaminophen Gel and Ointment Formulations

The in vitro skin permeation studies of the acetaminophen gel and ointment formulations described in Examples 1-3 were performed using the PermeGear Franz diffusion apparatus with the internal volume of 3 mL and orifice diameter of 9 mm. The dermatomed human cadaver skin (Anatomy Gifts Registry) was thawed at room temperature for 15 min. Skin pieces (2.25 cm²) are cut and placed in pH 7.4 PBS for 20 min to remove the glycerine used for cryopreservation of the cadaver skin. The skin pieces were blotted with tissue paper and placed between the donor and the receiver compartments. The receiver compartments were filled with 3 mL of pH 7.4 PBS and the skin was allowed to equilibrate with the receiving medium for 60 min, prior to drug dosing. Twenty microliters of the formulations were added to the donor compartment to form a thin film on the surface of the skin. The receiver compartment solution was maintained at 32±1° C. for a period of 24 hr. The control cells were dosed with 20 μL of saturated solution of APAP in pH 7.4 PBS. At 2, 4, 6, 8, 22, and 24 hrs, 0.5 mL of the receiver compartment solution was withdrawn and replaced with 0.5 mL of fresh buffer to maintain sink conditions. The drug content in the samples was analyzed using the HPLC method. The steady state flux (J) for permeation (μg/cm²/hr) was obtained from the slope of the linear potion of the plot of cumulative amount of APAP permeating per unit surface area of skin (μg/cm²) against time (hr). The permeability coefficient Kp was calculated from the ratio of the steady state flux (J) and the concentration in the donor compartment (Cd). Since saturated solutions were used as donor solutions, Cd was the saturation solubility of APAP for all practical purposes of the calculation. The enhancement in flux for skin permeation (EFflux) was calculated.

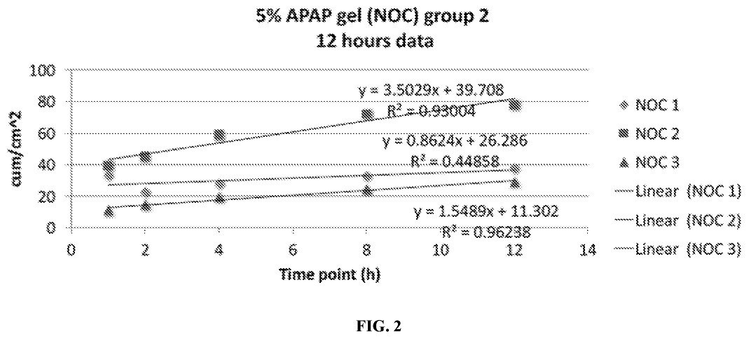

The results of the skin permeation studies are shown in FIGS. 2-7. When the Franz cell was covered, it is indicated as “occluded” in the figures. When the Franz cell was not covered, it is indicated as “non-occluded” in the figures.

FIG. 2 shows skin permeation obtained using 5% acetaminophen gel composition when tested using Franz cells not covered (non-occluded). Each symbol (square/diamond/triangle) represents individual Franz cell. The 5% gel, in non-occluded (NOC) conditions, provides about 10-40 μg/cm² of acetaminophen in the first hour.

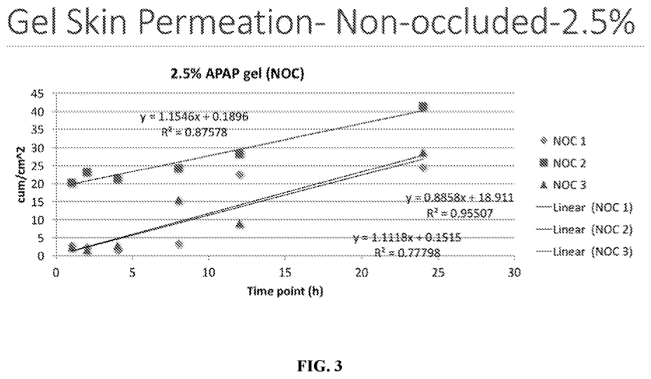

FIG. 3 shows skin permeation obtained using 2.5% acetaminophen gel composition when tested using Franz cells not covered (non-occluded). Each symbol represents individual Franz cell. The 2.5% gel, in non-occluded (NOC) conditions, provides about 2-20 μg/cm² of acetaminophen in the first hour.

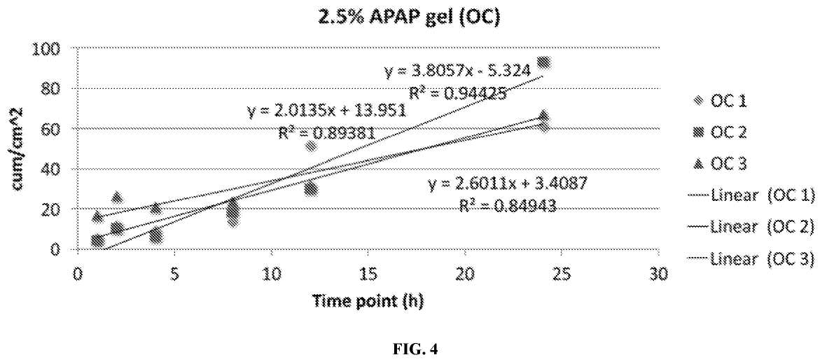

FIG. 4 shows skin permeation obtained using 2.5% acetaminophen gel composition when tested using Franz cells covered (occluded). Each symbol represents individual Franz cell. Occlusion of Franz cells does not appear to improve the initial permeation of acetaminophen; however, it may help with the longer term permeation.

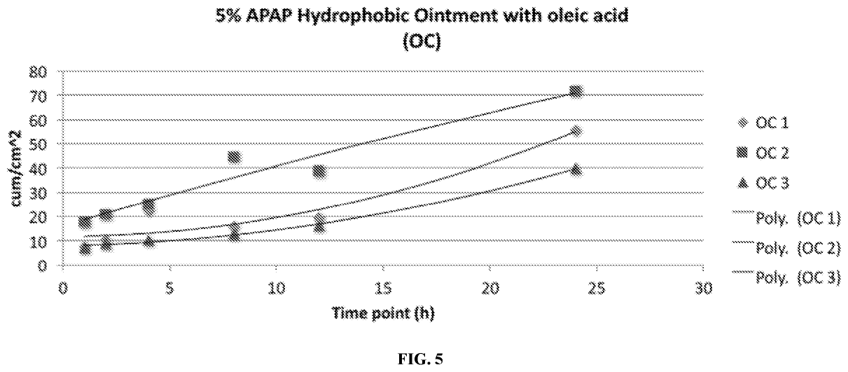

FIG. 5 shows skin permeation obtained using 5% acetaminophen hydrophobic ointment composition when tested using Franz cells covered (occluded). Each symbol represents individual Franz cell. The 5% hydrophobic ointment, with Franz cells occluded, provides about 2-20 μg/cm² of acetaminophen in the first hour. The permeation provided by this composition appears to be comparable to that provided by 2.5% acetaminophen gel composition, in non-occluded conditions.

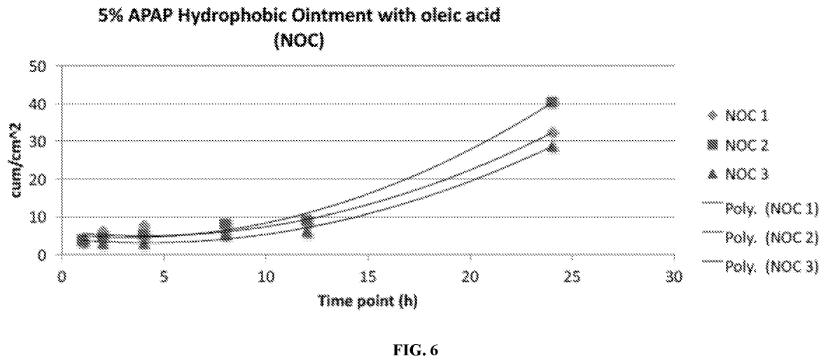

FIG. 6 shows skin permeation obtained using 5% acetaminophen hydrophobic ointment composition when tested using Franz cells not covered (non-occluded). Each symbol represents individual Franz cell. When Franz cells were not occluded, the ointment appears to provide low permeation. Low permeation could be due to a partitioning effect where the acetaminophen may not partition out of the dosage form into the skin.

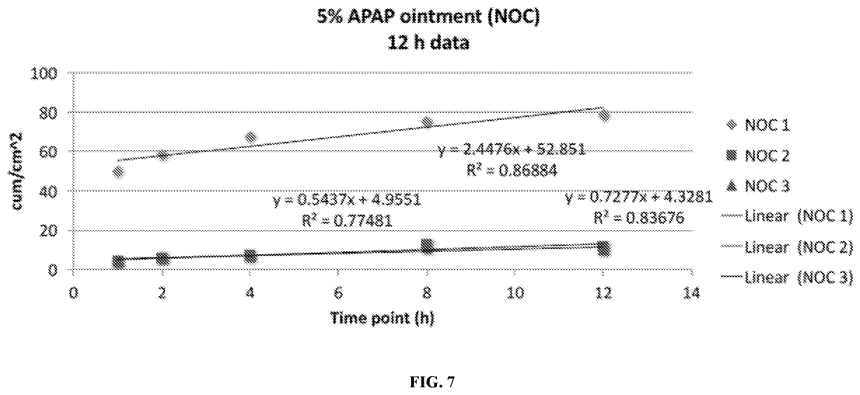

FIG. 7 shows skin permeation obtained using 5% acetaminophen hydrophilic base ointment composition when tested using Franz cells not covered (non-occluded). Each symbol represents individual Franz cell. There was a lot of variability amongst the samples. The 50 μg/cm² permeation value obtained with NOC 1 appears to be an error because the permeation as per the slope is very low. Low permeation could be due to the partitioning effect described above.

### Example 5: Finite Dose Skin Penetration Studies of Acetaminophen Gel and Ointment Formulations

The skin used in the finite dose permeation study described in Example 4 was further analyzed for drug content by separating the epidermis and the dermis using a heat separation method (Kassiv, V and Sondergaard, J., 1982 and Frank, J. D. et al 1995). A modification of the reported heat separation method is used for separation of epidermis and dermis. After the finite dose skin permeation experiment, the skin was dismounted from the Franz cell and washed with nanopure water thrice on both the epidermal and the dermal sides to remove any surface drug. The skin was then blotted with tissue paper; the area exposed to the drug was separated from the peripheral skin and wrapped in aluminum foil. Each of the samples was heated in the oven at 45° C. for 10 min. The epidermis was then peeled from the dermis using sharp forceps. The thickness of the epidermis and the dermis were measured using electronic digital micrometer (Marathon Management). The two layers were then placed in separate glass vials containing 1 mL of the mobile phase each at 37° C. for 24 hr to extract APAP. The concentration of the drug in the extracting medium was quantified using HPLC. The drug levels in Table 10 below are reported in μg/mL to determine the extent of absorption in the epidermis and the dermis. The enhancement factors, EF_{epidermis} and EF_{dermis} give the increase in accumulation of APAP in the epidermis and dermis relative to the control.

TABLE 10
Distribution of APAP after 24 hour treatment

| | | Amount | | | |
|---|---|---|---|---|---|
| Dosage Form | | (ug) | SD | %(of Dose) | SD |
| Gel | Epidermis | 121.9 | 30.0 | 41.5% | 9.17% |
| | Dermis | 85.7 | 9.3 | 29.4% | 4.21% |
| Oint#1 | Epidermis | 12.2 | 3.1 | 4.2% | 1.1% |
| | Dermis | 21.8 | 6.3 | 7.6% | 2.2% |
| Oint#2 | Epidermis | 106.1 | 48.3 | 35.1% | 15.1% |
| | Dermis | 69.2 | 29.7 | 22.9% | 9.2% |

### Example 6: Treatment of Chronic Itch

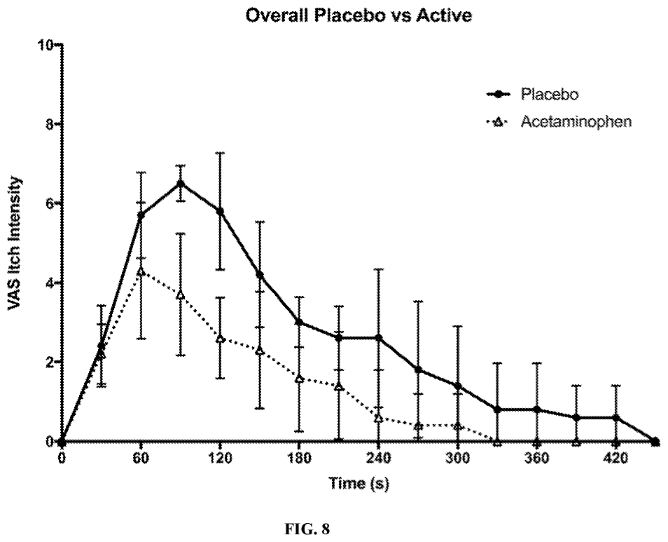

The efficacy of a topical acetaminophen composition was tested in 6 healthy subjects; itch was induced in the subjects by administering non-histaminergic itch stimuli which mimics chronic itch. The topical acetaminophen cream significantly reduced non-histaminergic itch over the vehicle cream (p=0.05) in healthy subjects. Peak itch relief was seen after 30 minutes of the topical application. During this time, the duration of itch was significantly reduced by 2 minutes and the intensity of the itch was reduced by 50% (p=0.03). See FIG. 8. Furthermore, redness was also reduced.

1. A topical composition comprising, a therapeutically effective amount of acetaminophen and a pharmaceutically acceptable excipient, wherein the acetaminophen is present in an amount from about 0.1% by weight to about 5% by weight, based on the total weight of the composition.

2. (canceled)

3. The topical composition of claim 1, comprising a solvent selected from the group consisting of water, alcohol, polyethylene glycol (PEG), methoxypolyethylene glycol (MPEG), poloxamers and mixtures thereof.

4. The topical composition of claim 3, wherein the alcohol is selected from the group consisting of ethanol, propylene glycol, and mixtures thereof.

5. The topical composition of claim 3, wherein the PEG is selected from the group consisting of PEG-300, PEG-400, and mixtures thereof.

6. (canceled)

7. The topical composition of claim 1, comprising a thickening agent.

8. The topical composition of claim 7, wherein the thickening agent is selected from the group consisting of hydroxypropylmethylcellulose (HPMC), carbomer, polyethylene glycol 6000, and mixtures thereof.

9. The topical composition of claim 1, comprising a neutralizing agent.

10. The topical composition of claim 9, wherein the neutralizing agent is triethanolamine.

11. The topical composition of claim 1, comprising an occlusive agent.

12. The topical composition of claim 11, wherein the occlusive agent is selected from the group consisting of paraffin, petrolatum, mineral oil, ceresin wax, and mixtures thereof.

13. The topical composition of claim 1, comprising a permeation enhancer.

14. The topical composition of claim 13, wherein the permeation enhancer is selected from the group consisting of oleic acid, propylene glycol, Laureth-4, N-methyl-pyrrolidone, Span 20, diethylene glycol mono ethyl ether, Lauryl-4, limonens, diethyl phthalate, triethyl citrate, triacetyl glycerin and bisabolol, and mixtures thereof.

15. The topical composition of claim 1, comprising an emulsifier.

16. The topical composition of claim 15, wherein the emulsifier is selected from the group consisting of esters of sorbitan.

17.-36. (canceled)

37. A method for treating itch in a patient in need thereof, comprising administering to the patient an effective amount of the topical composition of claim 1.

38.-39. (canceled)

40. The method of claim 37, wherein the itch is an acute itch or a chronic itch.

41. (canceled)

42. The method of claim 37, wherein the method provides peak itch relief about 5 minutes, 10 minutes, 15 minutes, 20 minutes, 25 minutes, 30 minutes, 35 minutes, 40 minutes, 45 minutes, 50 minutes, 55 minutes, or about 60 minutes, after the administration of the topical composition.

43. The method of claim 37, wherein the method provides a reduction in the duration of itch by about 2 minutes, 3 minutes, 4 minutes, 5 minutes, 6 minutes, 7 minutes, 8 minutes, 9 minutes, or about 10 minutes.

44. The method of claim 37, wherein the method provides a reduction in the intensity of itch by about 5%, 10%, 15%, 20%, 25%, 30%, 35%, 40%, 45%, 50%, 55%, 60%, 65%, 70%, 75%, or about 80%.

45. The method of claim 37, wherein the method provides a decrease in the redness of the skin.
